(19) United States
(12) Patent Application Publication
(10) Pub. No.: US 2023/0389813 A1
(43) Pub. Date: Dec. 7, 2023

(54) Estimating Heart Rate Recovery After Maximum or High-Exertion Activity Based on Sensor Observations of Daily Activities

(71) Applicant: Apple Inc., Cupertino, CA (US)

(72) Inventors: Britni A. Crocker, Santa Cruz, CA (US); Adeeti V. Ullal, Emerald Hills, CA (US); Ayse S. Cakmak, Santa Clara, CA (US); Johahn Y. Leung, San Francisco, CA (US); Katherine Niehaus, San Francisco, CA (US); William R. Powers, III, San Francisco, CA (US)

(21) Appl. No.: 17/952,147

(22) Filed: Sep. 23, 2022

Related U.S. Application Data
(60) Provisional application No. 63/348,851, filed on Jun. 3, 2022.

(51) Int. Cl. A61B 5/024; A61B 5/00

(52) U.S. Cl. CPC A61B 5/02438; A61B 5/6824; A61B 5/7264

(57) ABSTRACT

Embodiments are disclosed for estimating heart rate recovery (HRR) after maximum or high-exertion activity based on sensor observations. In some embodiments, a method comprises: obtaining, with at least one processor, sensor data from a wearable device worn on a wrist of a user; obtaining, with the at least one processor, a heart rate (HR) of the user; identifying, with the at least one processor, an observation window of the sensor data and HR; estimating, with the at least one processor during the observation window, input features for estimating maximum or near maximum exertion HRR of the user based on the sensor data and HR; and estimating, with the at least one processor during the observation window, the maximum or near maximum exertion HRR of the user based on a machine learning model and the input features.

### CROSS-REFERENCE TO RELATED APPLICATION

This application claims priority to U.S. Provisional Patent Application No. 63/348,851, filed Jun. 3, 2022, the entire contents of which are incorporated herein by reference.

### TECHNICAL FIELD

This disclosure relates generally to health monitoring and fitness applications.

### BACKGROUND

Heart rate recovery (HRR) after maximum or high-exertion exercise (hereinafter, referred to as “maximum exertion HRR”) has been shown to be predictive of long-term cardiovascular risk, and an independent predictor of cardiovascular fitness that is additive to measured quantities, such as maximal oxygen consumption (VO2 max). Maximum exertion HRR can also be used as a fitness indicator (e.g., indicative of overtraining).

Maximum exertion HRR may be quantified as a decrease in HR at certain time intervals (e.g., 1 minute, 3 minutes, etc.) after the completion of the activity. Existing methods for measuring maximum HRR are conducted in a laboratory setting, where the HR of an individual is measured and recorded immediately after maximum or high-exertion activity. The HR is measured and recorded again one minute later. The second HR measurement is subtracted from the first HR measurement to obtain the maximum exertion HRR. The larger the difference the more fit the individual and the lower their long-term cardiovascular risk.

### SUMMARY

Embodiments are disclosed for predicting maximum (or near maximum) exertion HRR based on sensor observations of daily activities. In some embodiments, a method comprises: obtaining, with at least one processor, sensor data from a wearable device worn on a wrist of a user; obtaining, with the at least one processor, a heart rate (HR) of the user; identifying, with the at least one processor, an observation window of the sensor data and HR; estimating, with the at least one processor during the observation window, input features for estimating maximum or near maximum exertion HRR of the user based on the sensor data and HR; and estimating, with the at least one processor during the observation window, the maximum or near maximum exertion HRR of the user based on a machine learning model and the input features.

Particular embodiments described herein provide one or more of the following advantages. The disclosed embodiments expand maximum or near maximum exertion HRR testing in a laboratory setting to an all-day setting, where users record exercise with a wearable device (e.g., smartwatch), but stop prior to or after actual indication of the end of a workout, where movement during and after activity is not controlled, where there is noise in sensor measurements and where observations may be made from low or high exertion exercise.

The details of one or more implementations of the subject matter are set forth in the accompanying drawings and the description below. Other features, aspects and advantages of the subject matter will become apparent from the description, the drawings and the claims.

### BRIEF DESCRIPTION OF THE DRAWINGS

FIG. 1 is a system for measuring maximum or near maximum exertion HRR, according to some embodiments.

FIG. 2 is a flow diagram of a process of measuring maximum or near maximum exertion HRR, according to some embodiments.

FIG. 3 is example system architecture implementing the features and operations described in reference to FIGS. 1-2.

### DETAILED DESCRIPTION

### Example System

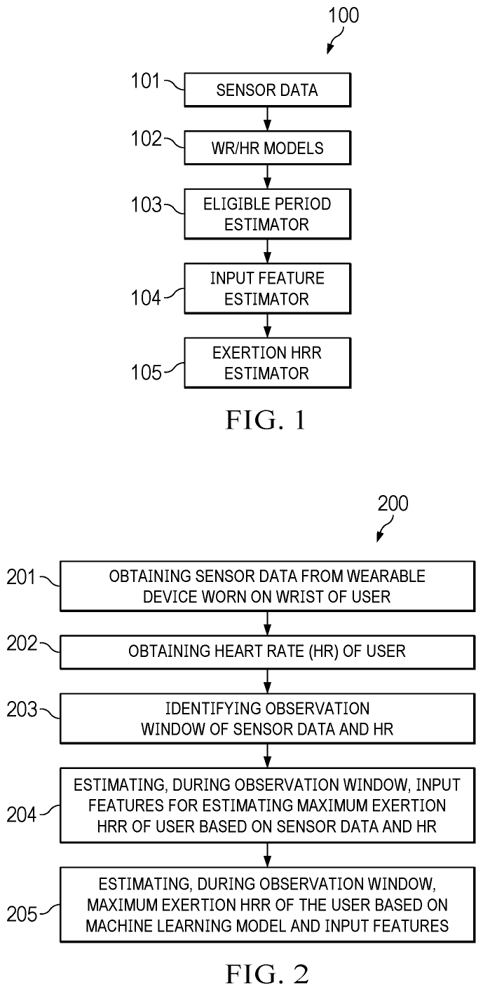

FIG. 1 is a system 100 for measuring maximum or near maximum exertion HRR, according to some embodiments. System 100 includes sensors 101, work rate (WR) and HR models 102, eligible period estimator 103, input feature estimator 104 and exertion HRR estimator 105. System 100 can be implemented on a wearable device, such as a smart watch or fitness band. Sensors 101 include inertial sensors (e.g., accelerometers, gyros), pressure sensor (e.g., a barometer), global navigation satellite system (GNSS) receiver (e.g., a GPS receiver) and HR sensor (e.g., a photoplethysmogram (PPG) sensor).

### I. Heart Rate/Work Rate Models

In some embodiments, the HR sensor outputs a current HR and confidence level that can be used to compute a normalized HR (NHR) or fraction of HR reserve (FHR). The NRH is input into an HR-based energy expenditure model 102 during exercise to estimate a current or “instantaneous” rate of energy expenditure during exercise. In some embodiments, the HR energy expenditure model takes the user's NHR as input and outputs the user's corresponding percentage of aerobic capacity (e.g., % VO2 max). If the user's individualized VO2 max has been previously calibrated, the HR energy expenditure model can convert % VO2 max into a metabolic equivalent of tasks (METs).

The WR energy expenditure model estimates a current or instantaneous rate of energy expenditure of the user during exercise based on motion data from the inertial sensors and other sensors (e.g., a barometer to measure grade). A confidence level may also be computed by the WR energy expenditure model. WR energy expenditure is dependent on the particular exercise being performed by the user. For example, if the user is running, the user's WR energy expenditure is a function of walking/running speed and grade.

The HR and WR energy expenditures are combined together to get a final user energy expenditure measurement. In some embodiments, HR and WR energy expenditures are combined or “fused” using a Bayesian probability formulation that determines a best estimate for energy expenditure given the WR and HR estimates of energy expenditure. In some embodiments, HR and WR energy expenditures are averaged. The best estimate of user energy expenditure is input into eligible period estimator 103.

### II. Eligible Period Estimator

Not all periods of potential observation can be used to infer HRR from maximum exertion using a machine learning model. For example, a minimal degree of exertion is desired to initiate a minimal drop in HR to enable maximum exertion estimator 104 to estimate (infer) a maximum exertion HRR. Valid periods for estimation should therefore meet certain criteria to be used for estimating maximum or near maximum exertion HRR. Some examples of eligibility criteria include but are not limited to: 1) eligible range of exertion, 2) period of steady-state recovery and 3) good sensor quality/confidence.

A. Eligible Range of Exertion

The exertion of the user during exercise should meet a minimal threshold to allow estimation of maximum or near maximum exertion HRR. Additionally, there should be a minimum recovery period post activity. Some example criteria that can be used to determine an eligible range of exertion include but are not limited to at least one of the following: personalized HR requirement, personalized HR drop during the recovery period, WR energy expenditure during exertion and closeness (shape-match) of the measured HRR trajectory with a prototypical HRR trajectory. In some embodiments, explained variance (e.g., coefficient of determination or R 2) is used to measure the discrepancy between the measured HRR trajectory and the prototypical HRR trajectory. Other embodiments may be used to measure discrepancy, such as root mean squared (RMS) error and time-weighted versions for R 2 and RMS.

An example criteria that can be used to determine a minimum recovery period is a minimum drop or proportional drop in WR energy expenditure post activity, which can be determined by comparing HR measured during the activity with HR measured post activity and comparing the difference to a specified minimum threshold. The drop can either be in terms of WR energy expenditure or HR (where HR is measured as NHR or FHR or HR).

B. Period of Steady State Recovery

When a user records a workout, they may actually stop activity several minutes before or after stopping the recording. To address this issue, the observation period should coincide with a period of steady state recovery, which can be determined by identifying the end of activity, determining a particular type of quiescence and identifying if the quiescence period has ended. In some embodiments, a period of steady state recovery is determined based on one or more of a threshold change in a smoothed (e.g., averaged) estimate of WR energy expenditure, a cross-correlation of a step function with the WR energy expenditure and user-recorded workout end time. An example quiescence period type is a period of inactivity or reduced activity with respect to a level of activity observed previously in an activity (e.g., a workout).

Additionally, there needs to be sufficient steady state recovery. Some example criteria to determine sufficient steady state recovery, include but are not limited to at least one of the following: sufficient period of time without passing a threshold on the amount of motion or number of steps, closeness (shape-match) of the measured HRR trajectory with a prototypical HRR trajectory based on an explained variance, similarity measure or other shape-match algorithm.

C. Sensor Quality/Confidence

Some example criteria for determining good quality HR measurements include but are not limited to at least one of the following: a minimum number of HR samples of good quality, a minimum number of samples within the observation window, goodness of fit of HR samples to a prototypical shape expectation and consistency of HR pre and post end of activity. Some example criteria for determining good quality WR measurements include but are not limited to at least one of the following: confidence in consistency of movement, confidence in sensor calibrations and confidence in types of movement. In some embodiments, the HR and motion sensors provide a confidence value. In some embodiments, consistency and confidence of movement can be estimated by, e.g., the standard deviation/variability of estimated mechanical WR, the change in rate of steps over time, the strength of signal for repetitive movement (versus chaotic movement) and the consistency of the mechanical work trajectory with the heart rate trajectory.

The output of eligible period estimator 103 are time demarcations of potential observation window(s) for predicting maximum exertion HRR. Sensor data that is captured within a potential observation window is provided to input features estimator 104, as well as sensor data outside the window (e.g., workload before the recovery period, HR after the recovery period).

### II. Input Features Estimator

Within a potential observation window, it is desirable to estimate various input features to make a valid prediction of maximum or near maximum exertion HRR. These input features are estimated by input feature estimator 104 and include but are not limited to one or more of the following examples: estimate of decrease in HR in the recovery period, estimate of recovery rate scaling factors, estimate of pre-recovery and during recovery load and estimate of steady state HR.

A. Estimate Decrease in HR in Observation Window

HR estimates are subject to noise and movement confounds, resulting in a need to estimate a truer notion of the HRR trajectory over the recovery period given these potential noise sources. In some embodiments, the noise can be filtered by smoothing the estimates of decrease in HR (e.g., using a median or mean filter), fitting a parameterized model (e.g., linear or non-linear regression model) to the smoothed estimates (e.g., sigmoid function, exponential curve) and computing a windowed mean of the smoothed estimates around the end of activity and at a specified number of subsequent observation windows.

B. Estimate of HRR Scaling Factors

The activity an individual is undertaking in a recovery period and the consistency of that activity will impact the rate of maximum or near maximum exertion HRR post activity. If an individual's HR is at steady-state for only part of the HRR period that should be identified and separated from subsequent parts of HRR. HRR scaling factors are a calculated correction to the observed decline in HR during the recovery period to estimate the decline that would have been observed during a maximal or high exertion activity.

In some embodiments, HRR scaling factors include but are not limited to at least one of: an estimate of consistency of activity using a threshold on WR energy expenditure, wrist movement and step variability; iteratively evaluate closeness of fit to a “model” HRR curve over consecutive windows; estimate of consistency of HR at the end of the observation window with HR in subsequent minutes; iteratively evaluated monotonicity of the HRR to find a minimum steady-state window.

C. Estimate of Pre-Recovery and During Recovery Loads

The expected rate of recovery may be a function of the degree of exertional “load” prior to the recovery period, and may similarly be blunted or accelerated depending on the degree of the exertional load during the recovery period. In some embodiments, estimates of pre-recovery and during recovery exertional loads include but are not limited to at least one of the following: the area under the curve of NHR and/or WR; maximum WR and/or HR achieved; maximum HR achieved; and the degree of variation in WR energy expenditure for the pre-recovery and during recovery periods.

D. Estimate of Steady-State HR

The steady-state HR achieved after a workout is indicative of the degree of fatigue induced by the workout and will inform the expected rate of recovery initially. In some embodiments, estimates of steady-state HR include but are not limited to at least one of: a percentile or threshold of HR in the recovery period, projection of HR into a future time, a best fit of steady-state HR from HRs in the post-recovery period and adjustment of observed HR for an observed level of activity.

The input features estimated by input feature estimator 104 over the observation window are input into exertion HRR estimator 105, which estimates maximum or near maximum exertion HRR using a machine learning model. In some embodiments, the machine learning model is a linear regression framework with the input features being the contributing features, or a parameterized physiological model where the modifying parameters are functions of the input features.

E. Estimate of HRR from User Information

In some embodiments, estimation of recovery from maximum or near maximum exertion includes the estimation of maximum or near maximum exertion, which can be estimated from a user's age, usage of medications, and historically observed measurements of HR.

### Example Process

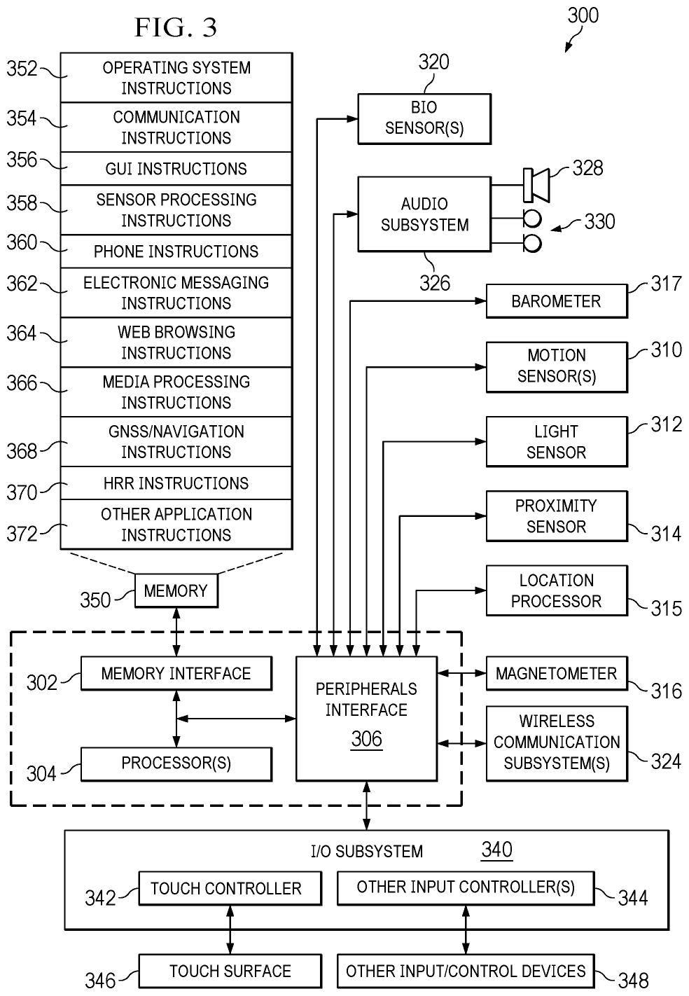

FIG. 2 is a flow diagram of a process for calculating maximum or near maximum exertion HRR, according to some embodiments. Process 200 can be implemented by, for example, using system architecture 300 described in reference to FIG. 3.

In some embodiments, process 200 includes: obtaining sensor data (201) from a wearable device worn on a wrist of a user; obtaining a heart rate (HR) of the user (202); identifying a potential observation window of sensor data and HR (203), estimating, during the observation window, input features for estimating maximum or near maximum exertion HRR of the user based on the sensor data and HR (204), and estimating, during the observation window, the maximum or near maximum exertion HRR of the user based on a machine learning model and the input features. Each of the steps was previously described in reference to FIG. 1.

### Exemplary System Architectures

FIG. 3 illustrates example system architecture 300 implementing the features and operations described in reference to FIGS. 1-2. Architecture 300 can include memory interface 302, one or more hardware data processors, image processors and/or processors 304 and peripherals interface 306. Memory interface 302, one or more processors 304 and/or peripherals interface 306 can be separate components or can be integrated in one or more integrated circuits. System architecture 300 can be included in any wearable device, including but not limited to: a smartwatch, fitness band, etc.

Sensors, devices and subsystems can be coupled to peripherals interface 306 to provide multiple functionalities. For example, one or more motion sensors 310, light sensor 312 and proximity sensor 314 can be coupled to peripherals interface 306 to facilitate motion sensing (e.g., acceleration, rotation rates), lighting and proximity functions of the mobile device. Location processor 315 can be connected to peripherals interface 306 to provide geo-positioning. In some implementations, location processor 315 can be a GNSS receiver, such as the Global Positioning System (GPS) receiver. Electronic magnetometer 316 (e.g., an integrated circuit chip) can also be connected to peripherals interface 306 to provide data that can be used to determine the direction of magnetic North. Electronic magnetometer 316 can provide data to an electronic compass application. Motion sensor(s) 310 can include one or more accelerometers and/or gyros configured to determine change of speed and direction of movement. Barometer 317 can be configured to measure atmospheric pressure, which can be used to determine altitude. Biosensors 320 can include a heart rate sensor, such as a photoplethysmography (PPG) sensor, electrocardiography (ECG) sensor, etc.

Communication functions can be facilitated through wireless communication subsystems 324, which can include radio frequency (RF) receivers and transmitters (or transceivers) and/or optical (e.g., infrared) receivers and transmitters. The specific design and implementation of the communication subsystem 324 can depend on the communication network(s) over which a mobile device is intended to operate. For example, architecture 300 can include communication subsystems 324 designed to operate over a GSM network, a GPRS network, an EDGE network, a Wi-Fi™ network and a Bluetooth™ network. In particular, the wireless communication subsystems 324 can include hosting protocols, such that the mobile device can be configured as a base station for other wireless devices.

Audio subsystem 326 can be coupled to a speaker 328 and a microphone 330 to facilitate voice-enabled functions, such as voice recognition, voice replication, digital recording and telephony functions. Audio subsystem 326 can be configured to receive voice commands from the user.

I/O subsystem 340 can include touch surface controller 342 and/or other input controller(s) 344. Touch surface controller 342 can be coupled to a touch surface 346. Touch surface 346 and touch surface controller 342 can, for example, detect contact and movement or break thereof using any of a plurality of touch sensitivity technologies, including but not limited to capacitive, resistive, infrared and surface acoustic wave technologies, as well as other proximity sensor arrays or other elements for determining one or more points of contact with touch surface 346. Touch surface 346 can include, for example, a touch screen or the digital crown of a smart watch. I/O subsystem 340 can include a haptic engine or device for providing haptic feedback (e.g., vibration) in response to commands from processor 304. In an embodiment, touch surface 346 can be a pressure-sensitive surface.

Other input controller(s) 344 can be coupled to other input/control devices 348, such as one or more buttons, rocker switches, thumb-wheel, infrared port and USB port. The one or more buttons (not shown) can include an up/down button for volume control of speaker 328 and/or microphone 340. Touch surface 346 or other controllers 344 (e.g., a button) can include, or be coupled to, fingerprint identification circuitry for use with a fingerprint authentication application to authenticate a user based on their fingerprint(s).

In one implementation, a pressing of the button for a first duration may disengage a lock of the touch surface 346; and a pressing of the button for a second duration that is longer than the first duration may turn power to the mobile device on or off. The user may be able to customize a functionality of one or more of the buttons. The touch surface 346 can, for example, also be used to implement virtual or soft buttons.

In some implementations, the mobile device can present recorded audio and/or video files, such as MP3, AAC and MPEG files. In some implementations, the mobile device can include the functionality of an MP3 player. Other input/output and control devices can also be used.

Memory interface 302 can be coupled to memory 350. Memory 350 can include high-speed random access memory and/or non-volatile memory, such as one or more magnetic disk storage devices, one or more optical storage devices and/or flash memory (e.g., NAND, NOR). Memory 350 can store operating system 352, such as the iOS operating system developed by Apple Inc. of Cupertino, California. Operating system 352 may include instructions for handling basic system services and for performing hardware dependent tasks. In some implementations, operating system 352 can include a kernel (e.g., UNIX kernel). In some embodiments, microphones 330 can be used to capture the breathing of the user, which can be used as an additional input/feature or derive an additional input/feature of a model for estimating HRR, as described in reference to FIG. 1.

Memory 350 may also store communication instructions 354 to facilitate communicating with one or more additional devices, one or more computers and/or one or more servers, such as, for example, instructions for implementing a software stack for wired or wireless communications with other devices, such as a sleep/wake tracking device. Memory 350 may include graphical user interface instructions 356 to facilitate graphic user interface processing; sensor processing instructions 358 to facilitate sensor-related processing and functions; phone instructions 360 to facilitate phone-related processes and functions; electronic messaging instructions 362 to facilitate electronic-messaging related processes and functions; web browsing instructions 364 to facilitate web browsing-related processes and functions; media processing instructions 366 to facilitate media processing-related processes and functions; GNSS/Location instructions 368 to facilitate generic GNSS and location-related processes and instructions; and instructions 370 that implement the features and processes described in reference to FIGS. 1 and 2. Memory 350 further includes application instructions 372 for performing various functions using, for example, estimating maximum HRR previously described in reference to FIGS. 1 and 2.

Each of the above identified instructions and applications can correspond to a set of instructions for performing one or more functions described above. These instructions need not be implemented as separate software programs, procedures, or modules. Memory 350 can include additional instructions or fewer instructions. Furthermore, various functions of the mobile device may be implemented in hardware and/or in software, including in one or more signal processing and/or application specific integrated circuits.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As described above, some aspects of the subject matter of this specification include gathering and use of data available from various sources to improve services a mobile device can provide to a user. The present disclosure contemplates that in some instances, this gathered data may identify a particular location or an address based on device usage. Such personal information data can include location-based data, addresses, subscriber account identifiers, or other identifying information.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

In the case of advertisement delivery services, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

What is claimed is:

1. A method comprising:
obtaining, with at least one processor, sensor data from a wearable device worn on a wrist of a user;
obtaining, with the at least one processor, a heart rate (HR) of the user;
identifying, with the at least one processor, an observation window of the sensor data and HR;
estimating, with the at least one processor during the observation window, input features for estimating maximum or near maximum exertion HR recovery (HRR) of the user based on the sensor data and HR; and
estimating, with the at least one processor during the observation window, the maximum or near maximum exertion HRR of the user based on a machine learning model and the input features.

2. A system comprising:
at least one processor;
memory storing instructions that when executed by the at least one processor, cause the at least one processor to perform the method recited in claim 1.

3. A non-transitory, computer-readable storage medium having stored thereon instructions that when executed by the at least one processor, causes the at least one processor to perform the method recited in claim 1.
